(19) United States
(12) Patent Application Publication
(10) Pub. No.: US 2017/0342086 A1
(43) Pub. Date: Nov. 30, 2017

(54) Compounds and methods for the treatment of drug resistance in cancer cells against paclitaxel

(71) Applicant: Ajay Kumar, San Juan, PR (US)

(72) Inventor: Ajay Kumar, San Juan, PR (US)

(21) Appl. No.: 15/013,616

(22) Filed: Feb. 2, 2016

Related U.S. Application Data
(63) Continuation-in-part of application No. 14/929,641, filed on Nov. 2, 2015.
(60) Provisional application No. 62/121,385, filed on Feb. 26, 2015.

(51) Int. Cl. C07D 491/153; G01N 21/65; A61K 31/337; G01N 33/574; A61K 31/4741

(52) U.S. Cl. CPC C07D 491/153; G01N 33/57496; A61K 31/4741; A61K 31/337; G01N 21/658; G01N 2333/47; G01N 2333/914

(57) ABSTRACT

The disclosure provides compounds and methods for treating cancer by inhibiting the formation of cancer cells resistant to paclitaxel by preventing the formation of GBP1:PIM1 protein interaction during a chemotherapeutic treatment. These compounds and methods are able to treat cancer individually or in conjunction with paclitaxel.

### RELATED APPLICATIONS

This application claims benefit of priority of provisional application 62/121,385 filed on Feb. 26, 2015, and of non-provisional application Ser. No. 14/929,641, filed on Nov. 2, 2015. Both applications are herein incorporated by reference.

### STATEMENT REGARDING FEDERALLY SPONSORED RESEARCH AND DEVELOPMENT

The study was supported by the National Institute of General Medical Sciences of the National Institutes of Health through grant no. 8 P20 GM 103475.

### BACKGROUND OF THE DISCLOSURE

### Field of the Disclosure

The present invention relates to compounds and methods of treating cancer using a GBP1:PIM1 inhibitor. Pharmaceutical compositions comprising a GBP1:PIM1 inhibitor in conjunction with paclitaxel is also provided.

Paclitaxel resistance is a problem of patients undergoing chemotherapy for various cancer types. This resistance undermines therapy outcome.

### Discussion of the Background

Resistance to chemotherapeutic drugs is one of the most important problems faced by some cancer patients. Cancer cells can develop certain resistance to a chemotherapeutic drug after the initial treatment (Bhattachary and Cabral, A Ubiquitous β-tubulin Disrupts Microtubule Assembly and Inhibits Cell Proliferation, Mol Biol Cell. 2004 July; 15(7): 3123-3131.2004). Many solid tumors are treated with microtubule targeted agents (MTAs). These drugs target the microtubules, essential structures for cell form and function. Treatment with MTAs results in resistance in many cases.

This resistance comes about by the expression of genes that code for alternate types of the tubulin protein, the building block of microtubules. Among these alternate types of tubulin, the βIII allows for development of resistance to MTAs.

This resistance is not only due to the tubulin subtypes but to complex protein interactions with microtubules such as PIM1 and GBP1 an GTPase incorporated to microtubules in the presence of βIII tubulin. It is therefore desirable to limit the influence of βIII tubulin in order to reinstate susceptibility to MTA drugs. Current targeted strategies for chemical treatment of cell malignancies include the use of small molecules that interact with the target proteins inhibiting their interactions and function. Podophyllotoxin (from Podophyllum spp.) and its derivatives have become interesting therapeutic choices for varied diseases or symptoms.

Aza-podophyllotoxin derivatives are synthetic scaffolds analogous to podophyllotoxin. This type of derivative has proven to be effective against cancer cells of various types (See, Vélez, Zayas and Kumar, Biological Activity of N-Hydroxyethyl-4-aza-2,3-didehydropodophyllotoxin

Derivatives upon Colorectal Adenocarcinoma Cells, Open Journal of Medicinal Chemistry, 2014, 4, 1-11; incorporated by reference herein). We have identified several APT's with potential anticancer activity in various tissues in a screening done at the National Cancer Institute's 60 cell line screening. Specifically, the novel APT NSC756093 (as per National Cancer Institute nomenclature) has proven effective experimentally as a target for the inhibition of GBP1:PIM1 interaction. (See, Kumar et al., Identification of the First Inhibitor of the GBP1:PIM1 Interaction. Implications for the Development of a New Class of Anticancer Agents against Paclitaxel Resistant Cancer Cells, J. Med. Chem., 2014, 57 (19), pp 7916-793; incorporated by reference herein). This compound has demonstrated high specificity to the target GBP1 protein and inhibits binding with PIM1 and conferring activity against paclitaxel resistant cancers.

### SUMMARY OF THE INVENTION

The present invention provides specific inhibitors of GBP1:PIM1 interaction. These inhibitors interfere with the protein-protein interaction thus preventing the resistance to paclitaxel conferred by these proteins. The present invention also relates to methods for preparation of compounds that inhibit GBP1:PIM1 complex formation. The present invention also relates to methods of treating cancer using a GBP1:PIM1 inhibitor.

In one embodiment, the invention relates to compounds for treating cancer by inhibiting a GBP1:PIM1 interaction which takes place in paclitaxel resistant cancers.

In another embodiment, the invention relates to a pharmaceutical composition as individual or for combination therapy purposes for treating cancer comprising inhibition of GBP1:PIM1 interaction which developed in various cancers during treatment with paclitaxel.

In another embodiment, the invention relates to a pharmaceutical composition for treating cancer comprising a GBP1:PIM1 inhibitor in conjunction with paclitaxel.

In another embodiment of this invention, the invention relates to compounds that inhibit the formation of GBP1:PIM1.

In further embodiments the invention relates to the compound 4-(2-Hydroxyethyl)-6-methoxy-9-phenyl-4,9-dihydrofuro[3,4-b]-quinolin-1(3H)-one, also known as compound NSC756093 as per National Cancer Institute register or APA 404.

In another embodiment, the invention relates to a pharmaceutical composition comprising 4-(2-Hydroxyethyl)-6-methoxy-9-phenyl-4,9-dihydrofuro[3,4-b]-quinolin-1(3H)-one and paclitaxel.

In another embodiment, the invention relates to a method of treating cancer using the compound 4-(2-Hydroxyethyl)-6-methoxy-9-phenyl-4,9-dihydrofuro[3,4-b]-quinolin-1(3H)-one as a GBP1:PIM1 inhibitor during a chemotherapy treatment.

In another embodiment, the invention relates to a method of treating cancer using the compound 4-(2-Hydroxyethyl)-6-methoxy-9-phenyl-4,9-dihydrofuro[3,4-b]-quinolin-1(3H)-one in conjunction with paclitaxel during a chemotherapy treatment.

In another embodiment of this invention, the invention relates to the use of these compounds as markers and inhibitor for βIII tubulin and GBP1 protein as well.

In another embodiment of this invention, the invention relates to the use of these compounds as markers for monitoring βIII tubulin expression in cancer patients during chemotherapy to monitor the progress of the treatment.

In another embodiment of this invention, the invention relates to the use of these compounds as markers for monitoring βIII tubulin expression immobilizing azapodophyllotoxin derivatives on biochip of surface plasmon resonance or any device to test GBP1 or B-III tubulin.

In another embodiment of this invention, the invention relates to the use of these compounds as markers for monitoring βIII tubulin expression for early detection of cancer.

In another embodiment of this invention, the invention relates to the use of these compounds as theranostics for various types of cancer.

### BRIEF DESCRIPTION OF THE DRAWINGS

The accompanying drawings, which are incorporated herein, constitute part of the specification and illustrate the preferred embodiment of the invention.

FIG. 1 shows the synthesis of compound 4-(2-Hydroxyethyl)-6-methoxy-9-phenyl-4,9-dihydrofuro[3,4-b]-quinolin-1(3H)-one, also known as NSC756093 or APA-404, in accordance with the principles of the present disclosure.

FIG. 2 shows the general synthesis of N-(2-hydroxyethyl)-2,3 didehydroazapodophyllotoxins, also known as Azapodophyllotoxin derivatives, in accordance with the principles of the present disclosure.

FIG. 3 shows the general structural formula of Azapodophyllotoxin derivatives according to the principles of the present invention.

FIG. 4 shows some Azapodophyllotoxin derivatives in accordance with the principles of the present disclosure.

FIG. 5 shows other Azapodophyllotoxin derivatives in accordance with the principles of the present disclosure.

FIG. 6 shows Representative coimmunoprecipitation (IP) of PIM1 and GBP1 in SKOV3 cell line treated with NSC756093 (3 h at 100 nM).

FIG. 7 shows a Bar chart showing the densitometric analysis of the experiment shown in FIG. 6, performed in two independent experiments.

FIG. 8 shows a plot chart of the results of Spearman correlation test between the Z-score of the tested active 4-APTs with carboplatin. In x- and y axis, the p value of the Spearman correlation and ρ values are plotted, respectively. Positive and negative ρ values indicate cross-sensitivity and cross-resistance, respectively.

FIG. 9 shows a plot chart of the results of Spearman correlation test between the Z-score of the tested active 4-APTs with paclitaxel. In x- and y axis, the p value of the Spearman correlation and ρ values are plotted, respectively. Positive and negative ρ values indicate cross-sensitivity and cross-resistance, respectively.

FIG. 10 shows a plot chart of the results of Spearman correlation test between the Z-score of the tested active 4-APTs in the NCI-60 cell lines. In x- and y axis, the p value of the Spearman correlation and ρ values are plotted, respectively. Positive and negative ρ values indicate cross-sensitivity and cross-resistance, respectively.

FIG. 11 is a Bar chart showing the % of inhibition of the compounds capable of producing an inhibition of the GBP1:PIM1 interaction >10% at a drug concentration of 100 nM. The maximum signal (100%) was obtained in the absence of any compound. Bar and error bars refer to mean and SD of duplicated experiments.

FIG. 12 is a Line chart reporting the dose dependent growth inhibition of NSC756093 and NSC756090. Each dot and bar refer to mean and SD of duplicated experiments.

FIG. 13 is a representative biosensograms for NSC756093. CA (dotted line) represented the negative control while GBP1 (red line) the maximum signal without inhibitor.

FIG. 14 is a representative biosensograms for NSC756090. CA (dotted line) represented the negative control while GBP1 (red line) the maximum signal without inhibitor.

### DETAILED DESCRIPTION OF THE INVENTION

The disclosure provides compounds and methods for treating cancer by inhibiting the formation of cancer cells resistant to paclitaxel by preventing the formation of GBP1:PIM1 complex on β-III tubulin protein during a chemotherapeutic treatment which expresses on tubulin protein during the treatment of cancer using tubulin inhibitor drugs. β-III tubulin overexpression is one of the important mechanisms of paclitaxel resistance with strong support in clinical studies. Additional synthesized compounds are described in patent application Ser. No. 14/929,641, filed on Nov. 2, 2015, incorporated herein by reference. In particular, these compounds and methods are able to treat cancer using a pharmaceutical composition comprising inhibition of GBP1:PIM1 complex formation, wherein paclitaxel can be effectively used at optimal amounts, and avoids the development of drug resistance i.e., amounts lower than the currently clinically recommended, thereby reducing the side effects of such treatment. The present compounds and methods overcome or reduce the risk of developing cancer cells resistant to tubulin inhibitor drug i.e. paclitaxel, which is one of the significant drawbacks of the chemotherapeutic therapy.

The present disclosure provides novel compound 4-(2-Hydroxyethyl)-6-methoxy-9-phenyl-4,9-dihydrofuro [3,4-b]-quinolin-1(3H)-one, also known as compound NSC756093 or APA-404, which is an Azapodophyllotoxin derivative, capable of inhibiting the formation of the GBP1:PIM1 complex by inhibiting binding site of GPB1, which over-expressed on tubulin protein to repair it against the treatment by paclitaxel and leads to develop drug resistance in cancer.

### Synthesis of Compound NSC756093

As shown in FIG. 1, a mixture of tetronic acid (326.27 mg, 3.13 mmol), substituted aniline 2-(3-methoxyphenylamino) ethanol (534.05 mg, 3.13 mmol) and benzaldehyde (319.4 μl, 3.13 mmol) dissolved in the minimum 2 ml of ethanol was irradiated under microwaves (300 watt, 100° C.); the reaction consumed approximately 6 watts during 45 minutes. After cooling, the precipitate was filtered off, washed with minimal cold ethanol and then recrystallized from ethanol, and characterized by UV, NMR and mass spectroscopy.

In another version of the synthesis, the mixture of tetronic acid (326.27 mg, 3.13 mmol), substituted aniline 2-(3-methoxyphenylamino)ethanol (534.05 mg, 3.13 mmol) and benzaldehyde (319.4 μl, 3.13 mmol) dissolved in 50:50 (Glycol:water) and irradiated under microwaves (300 watt, 100° C.); the reaction consumed approximately 6 watts during minutes. After cooling, the precipitate was filtered off, washed with minimal cold ethanol and then recrystallized from ethanol, and characterized by UV, NMR and mass spectroscopy. By changing solvent it becomes a total green synthetic method.

A different process was also used. In this process, after cooling, the precipitate is filtered off, slurry with dry silica gel 60, 230-400 mesh was prepared for column chromatography using silica gel 60, 230-400 mesh solid support. A mixture of Hexane: Ethyl Acetate: Acetonitrile (40:40:20) was used as eluent for purification of AP404 by silica column chromatography, and characterized by UV, NMR and mass spectroscopy.

Substituted aniline was synthesized as reported in our publication Synthesis of novel functionalized 4-aza-2,3-didehydropodophyllotoxin derivatives with potential antitumor activity, J. Het Chem. J. Heterocyclic Chem., Vol. 47, Issue 6, November (2010), pp. 1275-1282 (incorporated by reference herein).

Compound NSC756093, Yield: (564 mg, 51.29%), 1H NMR (DMSO-d6, 400 MHz): δ 3.67-3.71 (m, 3H), 3.73 (s, 3H), 3.81-3.92 (m, 1H), 4.93-5.16 (m, 4H), 6.56-6.59 (m, 1H), 6.70-6.71 (d, J=4 Hz, 1H), 6.96-7.01 (m, 1H), 7.12-7.18 (m, 1H), 7.20-7.28 (m, 4H). C NMR (DMSO-d6, 100 MHz): δ38.95, 47.85, 55.24, 57.77, 65.81, 96.27, 100.38, 108.62, 126.13, 127.54, 128.22, 132.05, 137.40, 158.68, 160.56, 172.0. LC-MS (ESI-TOF): ([C20H19 NO4+H]+ calcd m/z 338.1387) found 338.1382.

### GBP1 Inhibition

β-III tubulin is not suitable for in vitro screening due to the requirement of specific post-transcriptional confirmation in its structure. βIII-tubulin overexpresses GBP1 which enhances the binding a panel of pro-survival kinases like PIM1 this combination re-establishes the function of tubulin thus resistance develops.

At variance of βIII-tubulin, GBP1 and PIM1 can be expressed in vitro and can be used to test compounds capable of inhibiting protein:protein (GBP1:PIM1) interaction. The kinetics of the GBP1:PIM1 interaction was monitored with surface plasmon resonance technology (SPR). PIM1 was immobilized on the biochip as ligand and in a parallel flow path, carbonic anhydrase (CA) was immobilized on the chip as negative control. The binding of GBP1 was tested on both targets, flowing the protein on the chip surface at different concentrations. CA was also used as analyte control in the same range of [GBP1], thereby demonstrating that GBP1:PIM1 interaction is not dependent on any specific binding of PIM1.

All the analyses were performed in two independent channels of the biochip. Out of total Forty-four (44) Azapodophyllotoxin derivatives were tested, 32 compounds were completely inactive as inhibitor of the GBP1:PIM1 interaction. Eleven (11) compounds were capable of producing an inhibition of the binding around 10-20%. Compound NSC756093 was found capable of inhibiting 65% of the GBP1:PIM1 interaction.

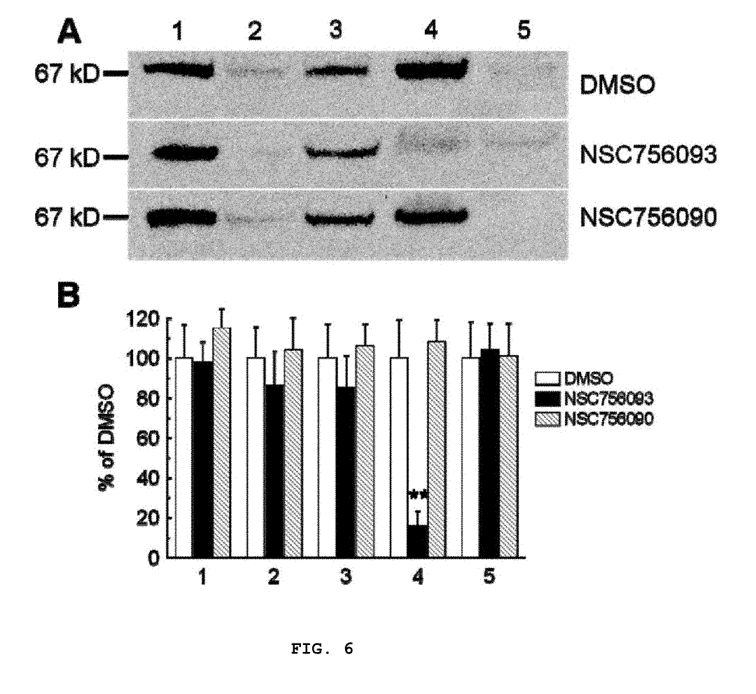

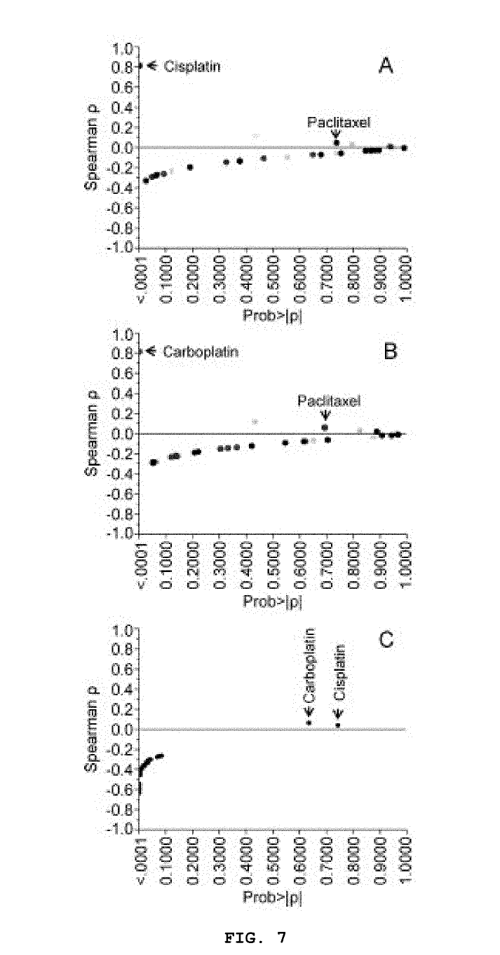

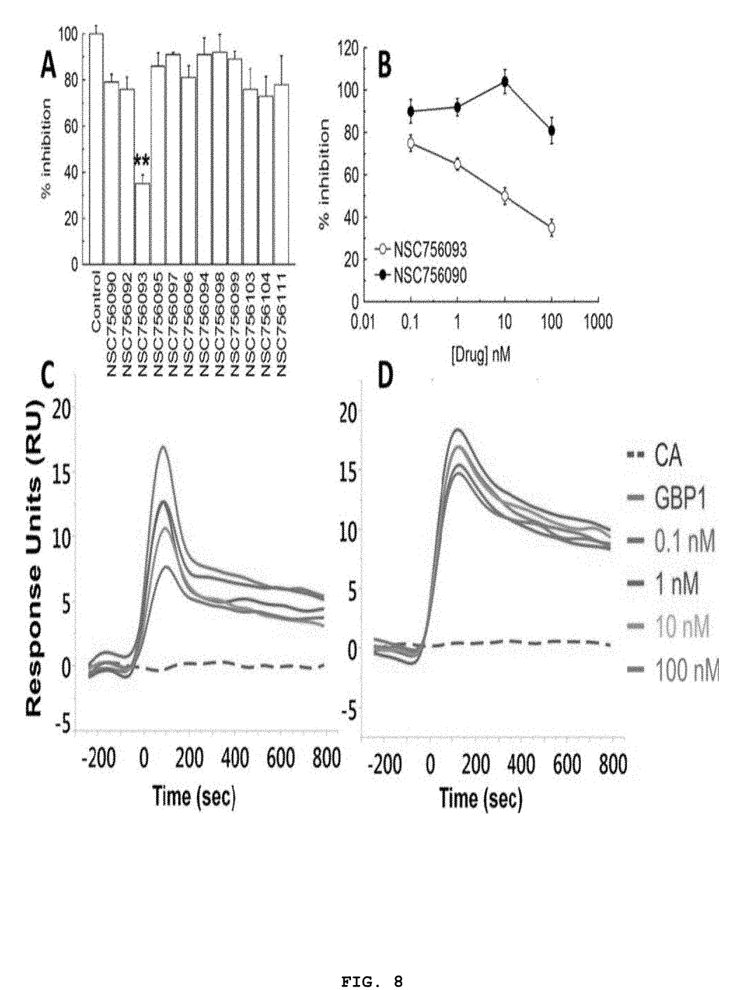

To confirm the activity of the drug in cell lines, the ability of NSC756093 was tested to inhibit the GBP1:PIM1 interaction in SKOV3 cells. The cells were treated for 3 h using 100 nM of the drug, then the cells were scraped and the pellet was used for coimmunoprecipitation of PIM1 with GBP1. The results demonstrated that treatment with NSC756093 inhibits the interaction of the GBP1:PIM1 interaction also in vitro as shown in FIG. 6 and FIG. 7.

### General synthesis of 4-aza-2,3-didehydropodophyllotoxin derivatives

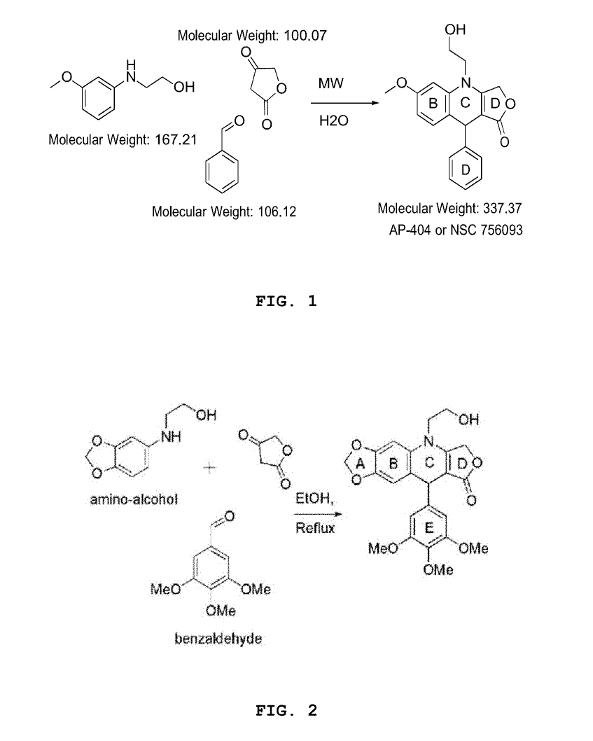

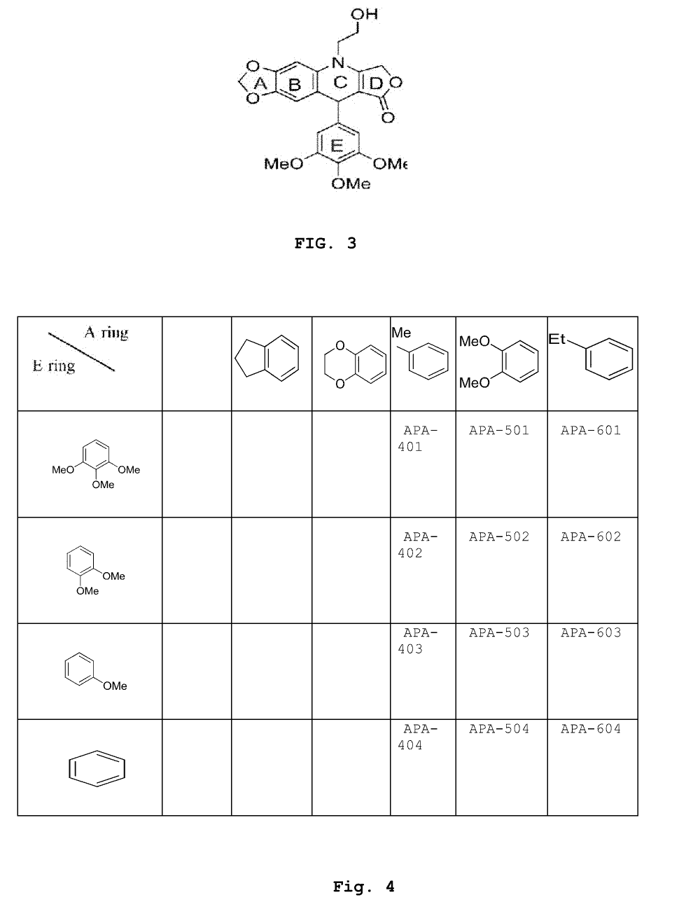

As shown in FIG. 2, an equimolar mixture of tetronic acid, substituted aniline and an aromatic aldehyde dissolved in the minimum volume of ethanol is irradiated with microwave (300 watt, 100° C.) using approximately 6 watts for 45 minutes. After cooling, the precipitate was filtered off, washed with minimal cold ethanol and then recrystallized from ethanol, and characterized by UV, NMR and mass spectroscopy.

In another version of the synthesis, the mixture of tetronic acid (326.27 mg, 3.13 mmol), substituted aniline 2-(3-methoxyphenylamino)ethanol (534.05 mg, 3.13 mmol) and benzaldehyde (319.4 μl, 3.13 mmol) dissolved in 50:50 (Glycol:water) and irradiated under microwaves (300 watt, 100° C.); the reaction consumed approximately 6 watts during 45 minutes. After cooling, the precipitate was filtered off, washed with minimal cold ethanol and then recrystallized from ethanol, and characterized by UV, NMR and mass spectroscopy. By changing solvent it becomes a total green synthetic method.

A different process was also used. In this process, after cooling, the precipitate is filtered off, slurry with dry silica gel 60, 230-400 mesh was prepared for column chromatography using silica gel 60, 230-400 mesh solid support. A mixture of Hexane: Ethyl Acetate: Acetonitrile (40:40:20) was used as eluent for purification of AP404 by silica column chromatography, and characterized by UV, NMR and mass spectroscopy.

Synthesis of substituted aniline is synthesized as reported in our publication S. Het Chem. J. Heterocyclic Chem., supra.

### Experimental Section

Chemistry. Melting points were determined on a MEL-TEMP instrument and are uncorrected. IR spectra were recorded on a PerkinElmer Spectrum 100 FTIR spectrometer on ATS mode. 1H, COSY, 13C, DEPT45, DEPT90, DEPT135, and HETCOR NMR spectra were measured on a Bruker 400 Ultra Shield spectrometer using DMSO-d6 as solvent. All chemical shifts are reported in parts per million relative to tetramethylsilane. Coupling constants (J) are reported in Hz. The LC-MS data was taken on an Agilent 1200 series system with Agilent 6210 time-of-flight mass detector. Absorption spectra were obtained in DMSO, using DMSO as blank, with an Agilent 8453 absorption spectrometer. The purities of all of the tested compounds were >95% as estimated by HPLC. General Synthesis of 4-Aza-2,3-didehydropodophyllotoxin Derivatives. These derivatives were synthesized by following previously reported method. An equimolar mixture of tetronic acid, substituted aniline, and aromatic aldehyde was dissolved in 50:50 (Glycol:water). The reaction mixture was refluxed for 30-90 min. After cooling, the precipitate was filtered off, washed with minimal cold ethanol, and then recrystallized from ethanol to afford the desired compound. Characterization data (NMR, HRMS etc.) of compounds NSC750210-750213, 750716-750723, and 751499-751504 have been published earlier.13,14 We found that the synthesis of products where R1 is methoxy at the meta position produced regioisomeric products with some aromatic aldehydes as observed from NMR data, while all remaining aryl amino alcohols produced only one regioisomeric expected product.

5-(2-Hydroxyethyl)-9-(3-chlorophenyl)-6,9-dihydro-[1,3]dioxolo-[4,5-g]furo[3,4-b]quinolin-8(5H)-one (NSC756083). Yield: 78%. 1H NMR (DMSO-d6, 400 MHz): δ 3.56-3.86 (m, 4H), 4.96 (t, 1H), 5.03-5.05 (d, J=8 Hz, 2H), 5.11 (s, 1H), 5.86-5.92 (m, 2H), 6.69 (s, 1H), 6.94 (s, 1H), 7.45-7.53 (m, 1H), 7.58-7.63 (m, 1H), 7.95-7.80 (m, 1H), 8.03-8.05 (m, 1H). 13C NMR (DMSO-d6, 100 MHz): δ39.36, 48.22, 57.89, 65.95, 94.07, 96.51, 101.50, 110.12, 117.83, 121.48, 121.90, 129.93, 131.25, 134.52, 143.47, 147.27, 147.79, 148.84, 160.81, 172.07. LC-MS (ESI-TOF): m/z 386.0770 ([C20H16 Cl NO5+H]+ calcd 386.0790).

4-(2-Hydroxyethyl)-10-(3-bromophenyl)-3,4,6,7,8,10-hexahydro-1H-cyclopenta[g]furo[3,4-b]quinolin-1-one (NSC756084). Yield: 70%. 1H NMR (DMSO-d6, 400 MHz): δ 1.90-2.00 (m, 2H), 2.61-2.77 (m, 2H), 2.80-2.84 (t, 2H), 3.65-3.78 (m, 3H), 3.80-3.92 (m, 1H), 4.98-5.06 (m, 2H), 5.10-5.17 (m, 2H), 6.92 (s, 1H), 7.11 (s, 1H), 7.17-7.25 (m, 2H), 7.30-7.38 (m, 1H), 7.42 (s, 1H). 13C NMR (DMSO-d6, 100 MHz): δ 25.13, 31.46, 32.33, 39.41, 48.23, 57.70, 65.84, 94.81, 110.17, 121.71, 123.84, 126.58, 126.87, 129.13, 130.19, 130.51, 134.81, 139.03, 143.73, 149.91, 160.82. LC-MS (ESI-TOF): m/z 426.0665 ([C22H20 Br NO3+H]+ calcd 426.0699).

4-(2-Hydroxyethyl)-10-(3-chlorophenyl)-3,4,6,7,8,10-hexahydro-1H-cyclopenta[g]furo[3,4-b]quinolin-1-one (NSC756085). Yield: 79%. 1H NMR (DMSO-d6, 400 MHz): δ 1.90-2.00 (m, 2H), 2.61-2.77 (m, 2H), 2.80-2.84 (t, 2H), 3.65-3.80 (m, 3H), 3.82-3.92 (m, 1H), 4.98-5.08 (m, 2H), 5.09-5.19 (m, 2H), 6.93 (s, 1H), 7.11 (s, 1H), 7.17-7.23 (m, 2H), 7.25-7.31 (m, 2H). 13C NMR (DMSO-d6, 100 MHz): δ 25.13, 31.46, 32.33, 39.43, 47.89, 57.70, 65.85, 94.80, 110.16, 123.84, 126.23, 126.44, 126.57, 127.37, 130.16, 132.96, 134.81, 139.02, 143.72, 149.65, 160.82. LC-MS (ESI-TOF): m/z 382.1206 ([C22H20 Cl NO3+H]+ calcd 382.1204).

4-(2-Hydroxyethyl)-10-(3,4-dichlorophenyl)-,4,6,7,8,10-hexahydro-1H-cyclopenta[g]furo[3,4-b]quinolin-1-one (NSC756086). Yield: 58%. 1H NMR (DMSO-d6, 400 MHz): δ 1.90-2.01 (m, 2H), 2.65-2.75 (m, 2H), 2.80-2.84 (t, 2H), 3.65-3.78 (m, 3H), 3.85-3.90 (m, 1H), 5.00-5.06 (m, 2H), 5.10-5.16 (m, 2H), 6.92 (s, 1H), 7.12 (s, 1H), 7.19-7.22 (m, 1H), 7.49-7.51 (m, 2H). 13C NMR (DMSO-d6,100 MHz): δ 25.13, 31.44, 32.33, 39.43, 47.91, 57.67, 65.92, 94.46, 110.27, 123.46, 126.57, 128.10, 128.91, 129.49, 130.51, 130.87, 134.77, 139.14, 143.89, 148.16, 160.95, 172.09. LC-MS (ESI-TOF): m/z 416.0803 ([C22H19 C12 NO3+H]+ calcd 416.0815).

6-(2-Hydroxyethyl)-10-(bromophenyl)-2,3,7,10-tetrahydro-[1,4]-dioxino[2,3-g]furo[3,4-b]quinolin-9(6H)-one (NSC756087). Yield: 70%. 1H NMR (DMSO-d6, 400 MHz): δ 3.62-3.75 (m, 3H), 3.76-3.85 (m, 1H), 4.13-4.18 (m, 2H), 4.18-4.26 (m, 2H), 4.94 (s, 1H), 5.02-5.18 (m, 3H), 6.57 (s, 1H), 6.76 (s, 1H), 7.18-7.25 (m, 2H), 7.31-7.38 (m, 1H), 7.43 (s, 1H). 13C NMR (DMSO-d6, 100 MHz): δ39.11, 48.03, 57.70, 63.95, 64.24, 65.84, 94.15, 103.11, 118.80, 118.87, 121.72, 126.76, 129.17, 130.13, 130.32, 130.51, 139.48, 142.62, 149.60, 160.65, 172.15. LC-MS (ESI-TOF): m/z 444.0404 ([C21H18 Br NO5+H]+ calcd. 444.0441).

6-(2-Hydroxyethyl)-10-(3-chlorophenyl)-2,3,7,10-tetrahydro-[1,4]dioxino[2,3-g]furo[3,4-b]quinolin-9(6H)-one (NSC756088). Yield: 61%. 1H NMR (DMSO-d6, 400 MHz): δ 3.62-3.75 (m, 3H), 3.76-3.85 (m, 1H), 4.13-4.18 (m, 2H), 4.18-4.26 (m, 2H), 4.95 (s, 1H), 5.00-5.17 (m, 3H), 6.57 (s, 1H), 6.76 (s, 1H), 7.16-7.24 (m, 2H), 7.25-7.32 (m, 2H). 13C NMR (DMSO-d6, 100 MHz): δ 39.14, 48.02, 57.70, 63.95, 64.24, 65.84, 94.14, 103.10, 118.79, 118.86, 126.26, 126.34, 127.29, 130.17, 130.33, 132.97, 139.48, 142.61, 149.34, 160.64, 172.14. LC-MS (ESI-TOF): m/z 400.0931 ([C21H18 Cl NO5+H]+ calcd 400.0946).

6-(2-Hydroxyethyl)-10-(3,4-dichlorophenyl)-2,3,7,10-tetrahydro-[1,4]dioxino[2,3-g]furo[3,4-b]quinolin-9(6H)-one (NSC756089). Yield: 70%. 1H NMR (DMSO-d6, 400 MHz): δ 3.62-3.83 (m, 4H), 4.08-4.30 (m, 4H), 4.93-5.20 (m, 4H), 6.58 (s, 1H), 6.78 (s, 1H), 7.19-7.21 (d, J=8 Hz, 1H), 7.50 (s, 2H). 13C NMR (DMSO-d6, 100 MHz): δ 38.15, 48.05, 57.69, 63.95, 64.25, 65.91, 93.84, 103.21, 118.44, 118.78, 127.98, 128.96, 129.41, 130.31, 130.50, 130.90, 139.57, 142.73, 147.83, 160.75, 172.12. LC-MS (ESI-TOF): m/z 456.0373 ([C21H17 Cl2 NO5+Na]+ calcd 456.0376).

4-(2-Hydroxyethyl)-6-methoxy-9-(3,4,5-trimethoxyphenyl)-4,9-dihydrofuro[3,4-b]quinolin-1(3H)-one (NSC756090). Yield: 54%. 1HNMR (DMSO-d6, 400 MHz): δ 3.58 (s, 3H), 3.65-3.72 (m, 10H), 3.74 (s, 3H), 4.85 (s, 1H), 4.98-4.52 (m, 1H), 5.06-5.19 (q, 2H), 6.51 (s, 2H), 6.59-6.62 (m, 1H), 6.71-6.72 (d, J=4 Hz, 1H), 7.11-7.13 (d, J=8 Hz, 1H). 13C NMR (DMSO-d6, 100 MHz): δ 38.86, 47.70, 55.23, 55.73, 57.85, 59.79, 65.83, 96.13, 100.35, 104.53, 108.64, 118.97, 131.82, 135.85, 137.07, 143.09, 152.72, 158.68, 160.83, 172.19. LC-MS (ESI-TOF): m/z 428.1699 ([C23H25 NO7+H]+ calcd 428.1704).

9-(3,4-Dimethoxyphenyl)-4-(2-hydroxyethyl)-6-methoxy-4,9-dihydrofuro[3,4-b]quinolin-1(3H)-one (NSC756091). Yield: 48%. 1H NMR (DMSO-d6, 400 MHz): δ 3.67-3.72 (m, 9H), 3.73 (s, 3H), 3.83-3.92 (m, 1H), 4.57-5.16 (m, 4H), 6.57-6.60 (m, 1H), 6.64-6.67 (m, 1H), 6.69-6.70 (d, J=J=4 Hz, 1H), 6.78-6.80 (d, J=8 Hz, 1H), 6.83-6.84 (d, J=4 Hz, 1H), 7.02-7.05 (m, 1H). 13C NMR (DMSO-d6, 100 MHz): δ 38.45, 47.76, 55.23, 55.38, 55.49, 57.81, 65.75, 96.41, 100.25, 108.57, 111.43, 111.81, 119.17, 119.28, 131.94, 137.20, 140.18, 147.21, 148.54, 158.60, 160.45, 172.18. LC-MS (ESITOF): m/z 398.1592 ([C22H23 NO6+H]+ calcd 398.1598).

4-(2-Hydroxyethyl)-6-methoxy-9-(3-methoxyphenyl)-4,9-dihydrofuro[3,4-b]quinolin-1(3H)-one (NSC756092). Yield: 52% [regio-isomeric mixture]. 1H NMR (400 MHz, acetonitrile-d3) δ3.74-3.67 (m, 1H), 3.75 (s, 3H), 3.79 (s, 3H), 3.91-3.79 (m, 4H), 4.95 (s, 1H), 5.10-4.97 (m, 2H), 6.59 (dd, J=8.5, 2.5 Hz, 1H), 6.65 (d, J=2.5 Hz, 1H), 6.75 (ddd, J=8.2, 2.6, 0.9 Hz, 1H), 6.81 (dd, J=2.6, 1.7 Hz, 1H), 6.85 (dt, J=7.6, 1.2 Hz, 1H), 7.04 (dd, J=8.4, 0.8 Hz, 1H), 7.21 (t, J=7.9 Hz, 1H). 13C NMR (101 MHz, CD3CN) δ40.44, 48.86, 55.71, 56.03, 59.37, 67.08, 101.29, 109.46, 112.32, 114.58, 120.03, 120.91, 130.38, 133.01, 138.59, 150.00, 160.17, 160.77, 161.48, 173.56. LC-MS (ESI-TOF): m/z 368.1491 ([C21H21 NO5+H]+ calcd 368.1492).

4-(2-Hydroxyethyl)-6-methoxy-9-phenyl-4,9-dihydrofuro[3,4-b]-quinolin-1(3H)-one (NSC756093). Yield: 50%. 1H NMR (DMSO-d6, 400 MHz): δ 3.67-3.71 (m, 3H), 3.73 (s, 3H), 3.81-3.92 (m, 1H), 4.93-5.16 (m, 4H), 6.56-6.59 (m, 1H), 6.70-6.71 (d, J=4 Hz, 1H), 6.96-7.01 (m, 1H), 7.12-7.18 (m, 1H), 7.20-7.28 (m, 4H). 13C NMR (DMSO-d6, 100 MHz): δ 38.95, 47.85, 55.24, 57.77, 65.81, 96.27, 100.38, 108.62, 126.13, 127.54, 128.22, 132.05, 137.40, 158.68, 160.56, 172.0. LC-MS (ESI-TOF): m/z 338.1382 ([C20H19 NO4+H]+ calcd 338.1387).

4-(2-Hydroxyethyl)-6-methoxy-9-(4-methoxyphenyl)-4,9-dihydrofuro[3,4-b]quinolin-1(3H)-one (NSC756094). Yield: 52% [regio-isomeric mixture]. 1H NMR (400 MHz, acetonitrile-d3) δ7.25-7.09 (m, 2H), 7.00 (dd, J=8.5, 0.8 Hz, 1H), 6.90-6.77 (m, 2H), 6.64 (d, J=2.4 Hz, 1H), 6.59 (dd, J=8.5, 2.5 Hz, 1H), 5.11-4.95 (m, 2H), 4.93 (s, 1H), 3.88-3.80 (m, 3H), 3.78 (s, 3H), 3.76 (s, 3H), 3.75-3.62 (m, 2H). 13C NMR (101 MHz, CD3CN): δ 172.71, 160.28, 159.17, 158.31, 139.88, 137.72, 132.18, 128.72, 119.63, 113.66, 108.59, 100.28, 97.40, 66.09, 58.50, 55.11, 54.83, 47.93, 38.69. LC-MS (ESI-TOF): m/z 368.1485 ([C21H21 NO5+H]+ calcd 368.1492).

9-(3-Bromophenyl)-4-(2-hydroxyethyl)-6-methoxy-4,9-dihydrofuro[3,4-b]quinolin-1(3H)-one (NSC756095). Yield: 51%. 1H NMR (CD3CN, 400 MHz): δ 3.79 (s, 3H), 3.80-3.82 (m, 4H), 4.82-5.03 (m, 4H), 6.59-6.62 (m, 1H), 6.55-6.66 (d, J=4 Hz, 1H), 6.98-7.00 (d, J=8 Hz, 1H), 7.22-7.27 (m, 2H), 7.34-7.38 (m, 1H), 7.42-7.43 (m, 1H). 13C NMR (CD3CN, 100 MHz): δ 38.95, 47.68, 54.83, 58.10, 65.93, 100.24, 108.46, 118.07, 121.74, 126.54, 129.14, 129.94, 130.02, 131.93, 137.50, 149.51, 159.13, 160.39, 172.23. LC-MS (ESITOF): m/z 438.0291 ([C20H18 Br NO4+Na]+ calcd 438.0311).

4-(2-Hydroxyethyl)-6,7-dimethoxy-9-(3,4,5-trimethoxyphenyl)-4,9-dihydrofuro[3,4-b]quinolin-1(3H)-one (NSC756097). Yield: 45%. 1H NMR (DMSO-d6, 400 MHz): δ 3.59 (s, 3H), 3.62 (s, 4H), 3.70 (s, 8H), 3.78 (s, 3H), 4.85 (s, 1H), 4.96-4.98 (t, 1H), 5.04-5.19 (m, 2H), 6.55 (s, 2H), 6.76 (s, 1H), 6.82 (s, 1H). 13C NMR (DMSO-d6,100 MHz): δ 39.54, 47.78, 55.75, 55.91, 55.96, 58.14, 59.80, 65.73, 94.70, 99.49, 104.49, 114.33, 118.15, 129.77, 135.84, 142.79, 145.12, 148.08, 152.72, 160.69, 172.39. LC-MS (ESI-TOF): m/z 458.1805 ([C24H27 NO8+H]+ calcd 458.1809). 9-(3,4-Dimethoxyphenyl)-4-(2-hydroxyethyl)-6,7-dimethoxy-4,9-dihydrofuro[3,4-b]quinolin-1(3H)-one (NSC756098). Yield: 64%. 1H NMR (DMSO-d6, 400 MHz): δ 3.61 (s, 3H), 3.68-3.69 (2s, 9H), 3.78 (s, 3H), 3.88-3.40 (m, 1H), 4.85 (s, 1H), 4.99-5.14 (m, 3H), 6.66-6.69 (m, 1H), 6.72 (s, 1H), 6.74 (s, 1H), 6.78-6.80 (d, J=8 Hz, 1H), 6.87-6.88 (d, J=4 Hz, 1H). 13C NMR (DMSO-d6, 100 MHz): δ38.90, 47.87, 55.40, 55.43, 55.86, 55.95, 58.11, 65.66, 95.03, 99.44, 111.38, 111.68, 114.42, 118.34, 119.20, 129.33, 139.88, 145.08, 147.20, 148.00, 148.53, 160.29, 172.36. LC-MS (ESI-TOF): m/z 428.1704 ([C23H25 NO7+H]+ calcd 428.1704).

4-(2-Hydroxyethyl)-6,7-dimethoxy-9-(3-methoxyphenyl)-4,9-dihydrofuro[3,4-b]quinolin-1(3H)-one (NSC756099). Yield: 68%. 1H NMR (DMSO-d6, 400 MHz): δ 3.60 (s, 3H), 3.69-3.73 (m, 5H), 3.79 (s, 3H), 3.87-3.98 (m, 1H), 4.90 (s, 1H), 5.00-5.15 (m, 3H), 6.68-6.73 (m, 2H), 6.76 (s, 1H), 6.78-6.81 (m, 2H), 7.13-7.17 (m, 1H). 13C NMR (DMSO-d6, 100 MHz): δ 39.38, 47.91, 54.84, 55.89, 55.95, 58.10, 59.72, 65.70, 94.84, 99.54, 111.19, 113.53, 114.48, 117.85, 119.77, 129.22, 130.11, 145.11, 148.12, 148.54, 159.20, 160.40, 172.28. LC-MS (ESI-TOF): m/z 398.1596 ([C22H23 NO6+H]+ calcd 398.1598).

4-(2-Hydroxyethyl)-6,7-dimethoxy-9-phenyl-4,9-dihydrofuro[3,4-b]quinolin-1(3H)-one (NSC756100). Yield: 60%. 1H NMR (DMSOd6, 400 MHz): δ 3.51 (s, 3H), 3.60-3.73 (m, 6H), 3.78-3.90 (m, 1H), 4.85 (s, 1H), 4.95-5.06 (m, 3H), 6.57 (s, 1H), 6.68 (s, 1H), 7.03-7.10 (m, 1H), 7.13-7.20 (m, 4H). 13C NMR (DMSO-d6, 100 MHz): δ 39.38, 47.91, 55.87, 55.95, 58.08, 65.71, 94.94, 99.55, 114.53, 117.96, 126.16, 127.50, 128.20, 130.17, 145.12, 146.98, 148.09, 160.36, 172.28. LC-MS (ESI-TOF): m/z 368.1490 ([C21H21 NO5+H]+ calcd 368.1492).

4-(2-Hydroxyethyl)-6,7-dimethoxy-9-(4-methoxyphenyl)-4,9-dihydrofuro[3,4-b]quinolin-1(3H)-one (NSC756102). Yield: 74%. 1H NMR (DMSO-d6, 400 MHz): δ 3.59 (s, 3H), 3.67-3.83 (m, 9H), 3.84-3.95 (m, 1H), 4.87 (s, 1H), 5.02-5.15 (m, 3H), 6.63 (s, 1H), 6.74 (s, 1H), 6.77-6.83 (m, 2H), 7.11-7.15 (m, 2H). 13C NMR (DMSO-d6, 100 MHz): δ 38.60, 47.89, 54.91, 55.88, 55.96, 58.08, 65.64, 95.19, 99.50, 113.55, 114.56, 118.35, 128.45, 130.12, 139.41, 145.11, 148.03, 157.61, 160.10, 172.30. LC-MS (ESI-TOF): m/z 398.1597 ([C22H23 NO6+H]+ calcd 398.1598).

9-(3-Bromophenyl)-4-(2-hydroxyethyl)-6,7-dimethoxy-4,9-dihydrofuro[3,4-b]quinolin-1(3H)-one (NSC756103). Yield: 70%. 1H NMR (DMSO-d6, 400 MHz): δ 3.60 (s, 3H), 3.68-3.78 (m, 2H), 3.79 (s, 4H), 3.85-3.95 (m, 1H), 4.98 (s, 1H), 5.02-5.16 (m, 3H), 6.67 (s, 1H), 6.77 (s, 1H), 7.18-7.25 (m, 2H), 7.32-7.38 (m, 1H), 7.41-7.45 (m, 1H). 13C NMR (DMSO-d6, 100 MHz): δ 39.26, 47.97, 55.93, 58.02, 65.82, 94.38, 99.64, 114.48, 117.12, 121.68, 126.76, 129.14, 130.14, 130.19, 130.45, 145.22, 148.29, 149.48, 160.61, 172.20. LC-MS (ESI-TOF): m/z 446.0528 ([C21H20 Br NO5+H]+ calcd 446.0598).

9-(3-Chlorophenyl)-4-(2-hydroxyethyl)-6,7-dimethoxy-4,9-dihydrofuro[3,4-b]quinolin-1(3H)-one (NSC756104). Yield: 72%. 1H NMR (DMSO-d6, 400 MHz): δ 3.60 (s, 3H), 3.68-3.77 (m, 2H), 3.79 (s, 4H), 3.87-3.95 (m, 1H), 4.99 (s, 1H), 5.02-5.16 (m, 3H), 6.67 (s, 1H), 6.77 (s, 1H), 7.18-7.25 (m, 2H), 7.26-7.30 (m, 2H). 13C NMR (DMSO-d6, 100 MHz): δ 39.26, 47.97, 55.92, 58.03, 65.83, 94.37, 99.64, 114.45, 117.13, 126.25, 126.35, 127.30, 130.11, 130.18, 132.93, 145.22, 148.28, 149.24, 160.62, 172.21. LC-MS (ESI-TOF): m/z 402.1080 ([C21H20 Cl NO5+H]+ calcd 402.1103).

9-(3,4-Dichlorophenyl)-4-(2-hydroxyethyl)-6,7-dimethoxy-4,9-dihydrofuro[3,4-b]quinolin-1(3H)-one (NSC756105). Yield: 71%. 1H NMR (DMSO-d6, 400 MHz): δ 3.60 (s, 3H), 3.68-3.77 (m, 2H), 3.79 (s, 4H), 3.87-3.95 (m, 1H), 4.99 (s, 1H), 5.02-5.06 (m, 2H), 5.08-5.15 (m, 2H), 6.67 (s, 1H), 6.77 (s, 1H), 7.19-7.22 (m, 1H), 7.48-7.53 (m, 2H). 13C NMR (DMSO-d6, 100 MHz): δ 38.55, 47.98, 55.92, 58.00, 65.90, 94.06, 99.69, 114.37, 116.70, 128.00, 128.88, 129.42, 130.14, 130.46, 130.83, 145.29, 147.74, 148.37, 160.73, 172.18. LC-MS (ESI-TOF): m/z 436.0646 ([C21H19 Cl2 NO5+H]+ calcd 436.0713).

6-Ethyl-4-(2-hydroxyethyl)-9-(3,4,5-trimethoxyphenyl)-4,9-dihydrofuro[3,4-b]quinolin-1(3H)-one (NSC756106). Yield: 54%. 1HNMR (DMSO-d6, 400 MHz): δ 1.14-1.18 (t, 3H), 2.53-2.59 (q, 2H), 3.59-3.72 (m, 12H), 3.95-4.02 (m, 1H), 4.88 (s, 1H), 4.99 (bs, 1H), 5.06-5.21 (q, 2H), 6.53 (s, 2H), 6.83-6.85 (d, J=8 Hz, 1H), 7.04 (s, 1H), 7.12-7.14 (d, J=8 Hz, 1H). 13C NMR (DMSO-d6, 100 MHz): δ 15.60, 28.00, 38.86, 47.55, 55.73, 57.83, 59.79, 65.82, 95.52, 104.56, 113.40, 122.96, 124.02, 130.90, 135.88, 142.96, 143.29, 152.74, 160.98, 172.25. LC-MS (ESI-TOF): m/z 426.1915 ([C24H27 NO6+H]+ calcd 426.1911).

6-Ethyl-4-(2-hydroxyethyl)-9-phenyl-4,9-dihydrofuro[3,4-b]-quinolin-1(3H)-one (NSC756108). Yield: 56%. 1H NMR (DMSO-d6, 400 MHz): δ 1.06-1.10 (t, 3H), 2.45-2.51 (q, 2H), 3.58-3.70 (m, 3H), 3.75-3.3 (m, 1H), 4.88 (s, 1H), 4.96-4.50 (m, 1H), 5.02-5.09 (m, 2H), 6.73-6.75 (m, 1H), 6.90-6.92 (d, J=8 Hz, 1H), 6.95 (s, 1H), 7.04-7.08 (m, 1H), 7.12-7.17 (m, 4H). 13C NMR (DMSO-d6,100 MHz): δ 15.58, 27.98, 38.94, 47.69, 57.74, 65.81, 95.67, 113.42, 122.92, 123.90, 126.17, 127.58, 128.25, 131.14, 136.21, 143.32, 147.14, 160.70, 172.16. LC-MS (ESI-TOF): m/z 336.1599 ([C21H21 NO3+H]+ calcd 336.1594).

9-(3-Bromophenyl)-6-ethyl-4-(2-hydroxyethyl)-4,9-dihydrofuro-[3,4-b]quinolin-1(3H)-one (NSC756110). Yield: 55%. 1H NMR (DMSO-d6, 400 MHz): δ 1.14-1.18 (t, 3H), 2.54-2.59 (q, 2H), 3.65-3.82 (m, 3H), 3.84-3.92 (m, 1H), 5.01-5.19 (m, 4H), 6.83-6.85 (m, 1H), 6.98-7.00 (d, J=8 Hz, 1H), 7.05 (s, 1H), 7.21-7.25 (m, 2H), 7.33-7.36 (m, 1H), 7.43 (s, 1H). 13C NMR (DMSO-d6, 100 MHz): δ 15.53, 27.98, 38.90, 47.76, 57.70, 65.93, 95.08, 113.63, 121.71, 123.09, 123.15, 126.89, 129.17, 130.22, 130.51, 131.17, 136.19, 143.64, 149.62, 160.98, 172.09. LC-MS (ESI-TOF): m/z 414.0626 ([C21H20 Br NO3+H]+ calcd 414.0699).

9-(3-Chlorophenyl)-6-ethyl-4-(2-hydroxyethyl)-4,9-dihydrofuro-[3,4-b]quinolin-1(3H)-one (NSC756111). Yield: 53%. 1H NMR (DMSO-d6, 400 MHz): δ 1.14-1.18 (t, 3H), 2.54-2.58 (q, 2H), 3.68-3.80 (m, 3H), 3.80-3.91 (m, 1H), 5.03-5.19 (m, 4H), 6.83-6.85 (d, J=8 Hz, 1H), 6.99-7.01 (d, J=8 Hz, 1H), 7.05 (s, 1H), 7.18-7.23 (m, 2H), 7.25-7.30 (m, 2H). 13C NMR (DMSO-d6, 100 MHz): δ 16.02, 28.49, 39.44, 48.26, 58.20, 66.43, 95.58, 114.12, 123.57, 123.65, 126.76, 126.97, 127.90, 130.66, 131.65, 133.48, 136.69, 144.14, 149.86, 161.48, 172.60. LC-MS (ESI-TOF): m/z 370.1170 ([C21H20 Cl NO3+H]+ calcd 370.1204).

9-(3,4-Dichlorophenyl)-6-ethyl-4-(2-hydroxyethyl)-4,9-dihydrofuro[3,4-b]quinolin-1(3H)-one (NSC756112). Yield: 70%. 1H NMR (DMSO-d6, 400 MHz): δ 1.14-1.18 (t, 3H), 2.54-2.59 (q, 2H), 3.69-3.81 (m, 3H), 3.83-3.91 (m, 1H), 5.03-5.19 (m, 4H), 6.83-6.85 (m, 1H), 6.99-7.01 (d, J=8 Hz, 1H), 7.05 (s, 1H), 7.18-7.22 (m, 2H), 7.28-7.30 (m, 1H). 13C NMR (DMSO-d6, 100 MHz): δ 15.53, 27.99, 38.94, 47.76, 57.70, 65.93, 95.08, 113.63, 123.07, 123.15, 126.26, 126.47, 127.40, 130.16, 131.15, 132.98, 136.19, 143.63, 149.36, 160.98, 172.10. LC-MS (ESI-TOF): m/z 404.0711 ([C21H19Cl2 NO3+H]+ calcd 404.0815).

As discussed in our recent manuscript in J. Med. Chem., 2014, 57 (19), pp 7916-793, supra, and based on Structure Activity Relationships (SAR), NSC756093 is the sterically less hindered structure of the series as compared to all other analogues presenting bulkier substituents at B and E rings, and it is also the only tested azapodophyllotoxin derivative which resulted in being able to significantly inhibit GBP1:PIM1 complex formation.

According to the principles of the present invention, other azapodophyllotoxin derivatives can be synthesized which have less bulky substituents at ring B and ring E of azapodophyllotoxin derivatives. These compounds will work as GBP1:PIM1 complex inhibitor, B-II tubulin inhibitor and as markers to monitor the progress of treatment or to make testing kits for over-expression of GBP1 for early detection of cancer.

Some modifications in the active structure of the azapodophyllotoxin derivatives are possible according to the principles of the present invention. For example, no side chain at the place of ethyl alcohol or all kind of side substituted or non-substituted chains in place of ethyl alcohol:

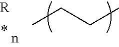

Where n is number 1, 2, 3 . . . so on. R is substituted or any functionalized hydroxyl, amine, acid, aldehyde, amino acid, fatty chain, alkyl chain, or sugar etc.

Close analogues of the APA 404 which will show similar effect are: ring B and ring E as substituted or substituted phenyl, substituted or substituted heterocyclic ring of 6 to 4 atoms for example: pyridine, pyridazine, pyrimidine, pyrazine, 1,2,3-triazine, oxazole, isoxazole, thiazole, isothiazole, 1H-pyrazole, and 1H-imidazole.

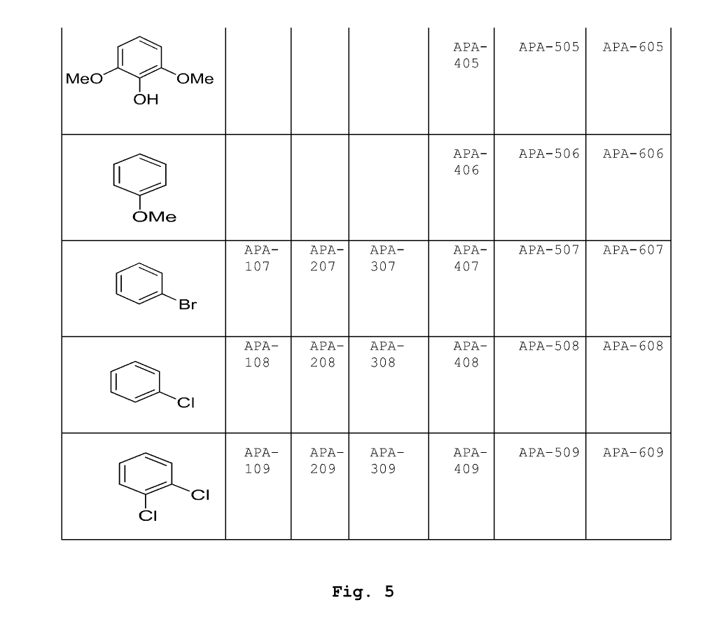

Close analogues of the APA 404 according to the principles of the present invention include, but are not limited to, compounds as described in FIGS. 4-5.

It should be apparent from consideration of the above illustrative examples that numerous exceptionally valuable products and processes are provided by the present disclosure in its many aspects.

Viewed in light, therefore, the specific disclosures of illustrative examples are clearly not intended to be limiting upon the scope. Numerous modifications and variations are expected to occur to those skilled in the art.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to limit the breadth of the application, which is measured by the claims, nor is it intended to be limiting as to the scope of the disclosure in any way.

What is claimed:

1. Azapodophyllotoxin compositions having the general formula: 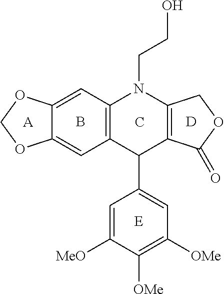
wherein a compound is selected from the group consisting of APA-107, APA-108, APA-109, APA-207, APA-208, APA-209, APA-307, APA-308, APA-309, APA-401, APA-402, APA-403, APA404, APA-405, APA-406, APA-407, APA-408, APA-409, APA-501, APA-502, APA-503, APA-504, APA-505, APA-506, APA-507, APA508, APA-509, APA-601, APA-602, APA-603, APA-604, APA-605, APA-606, APA-607, APA-608, and APA-609, as shown in FIG. 4 of the drawings.

2. The Azapodophyllotoxin composition of claim 1, wherein the selected compound is APA-404.

3. In combination, the composition according to claim 1 and paclitaxel.

4. A method for treating cancer using a GBP1:PIM1 inhibitor comprising:
administering to a subject an effective amount of at least one Aza-podophyllotoxin derivative of general formula: 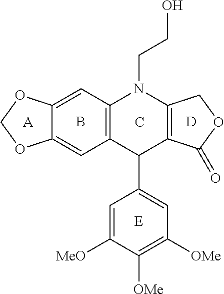
wherein A-ring is selected from the group consisting of 1,3-dioxolane, cyclopentane, 1,4-dioxane, one methoxy, two methoxys, and ethyl; and wherein E-ring is selected from the group consisting of dimethoxyanisole, veratrol, anisole, benzene, syringol, bromobenzene, chlorobenzene, 1,2-dichlorobenzene, 2,3-dimethoxybenzene, 3,4,5-trimethoxybenzene.

5. The method according to claim 4, wherein the at least one Aza-podophyllotoxin derivative is APA404.

6. The method according to claim 4, wherein the at least one Aza-podophyllotoxin derivative is administered to the subject in a dose of 100 nM.

7. The method according to claim 5, wherein APA404 is administered to the subject in a dose of 100 nM.

8. The method according to claim 5, wherein paclitaxel is administered to the subject.

9. The method according to claim 7, wherein an effective amount of paclitaxel is administered to the subject.

10. A method of marking proteins comprising:
selecting a protein for marking from the group consisting of βIII tubulin and GBP1; and
administering at least one Aza-podophyllotoxin derivative of general formula: 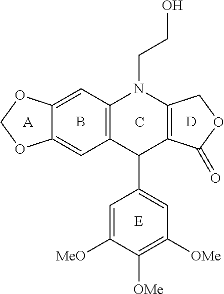
wherein A-ring is selected from the group consisting of 1,3-dioxolane, cyclopentane, 1,4-dioxane, one methoxy, two methoxys, and ethyl; and wherein E-ring is selected from the group consisting of dimethoxyanisole, veratrol, anisole, benzene, syringol, bromobenzene, chlorobenzene, 1,2-dichlorobenzene, 2,3-dimethoxybenzene, 3,4,5-trimethoxybenzene.

11. The method according to claim 10, wherein the at least one Aza-podophyllotoxin derivative is APA404.

12. The method according to claim 10, wherein the at least one Aza-podophyllotoxin derivative is administered to the subject in a dose of 100 nM.

13. The method according to claim 11, wherein APA404 is administered to the subject in a dose of 100 nM.

14. The method according to claim 11, wherein the selected protein is βIII tubulin.

15. The method according to claim 14, wherein administration is performed on a cancer patient to monitor βIII tubulin expression during chemotherapy.

16. The method according to claim 10, wherein the Aza-podophyllotoxin derivative is immobilized on a biochip to test for GBP1 or βIII tubulin.

17. The method of claim 16, wherein surface plasmon resonance is applied to the biochip.

18. A method comprising:
administering to a subject an effective amount of at least one Aza-podophyllotoxin derivative of general formula: 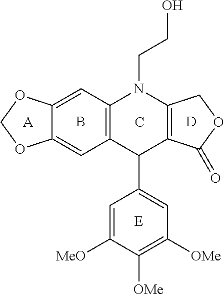
wherein A-ring is selected from the group consisting of 1,3-dioxolane, cyclopentane, 1,4-dioxane, one methoxy, two methoxys, and ethyl; and wherein E-ring is selected from the group consisting of dimethoxyanisole, veratrol, anisole, benzene, syringol, bromobenzene, chlorobenzene, 1,2-dichlorobenzene, 2,3-dimethoxybenzene, 3,4,5-trimethoxybenzene;
for inhibiting the events selected from the group consisting of protein-protein interaction and protein-protein formation.

19. The method according to claim 18, wherein such protein-protein is GBP1:PIM1.

20. The method according to claim 19, wherein the at least one Aza-podophyllotoxin derivative is APA404.

21. The method according to claim 20 wherein APA404 is administered in a dose of 100 nM.

22. The method according to claim 21, wherein such inhibition prevents resistance to paclitaxel.

23. A method comprising:
administering to a subject an effective amount of at least one Aza-podophyllotoxin derivative of general formula: 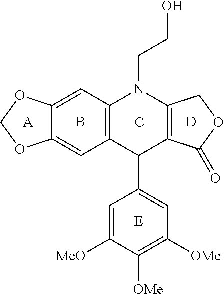
wherein A-ring is selected from the group consisting of 1,3-dioxolane, cyclopentane, 1,4-dioxane, one methoxy, two methoxys, and ethyl; and wherein E-ring is selected from the group consisting of dimethoxyanisole, veratrol, anisole, benzene, syringol, bromobenzene, chlorobenzene, 1,2-dichlorobenzene, 2,3-dimethoxybenzene, 3,4,5-trimethoxybenzene;
for inhibiting the events selected from the group consisting of protein-protein interaction and protein-protein formation.

24. The method according to claim 23, wherein such protein-protein is GBP1:PIM1.

25. The method according to claim 24, wherein the at least one Aza-podophyllotoxin derivative is APA404.

26. The method according to claim 25 wherein APA404 is administered in a dose of 100 nM.

27. The method according to claim 26, wherein such inhibition prevents resistance to paclitaxel.

28. A synthesis procedure for a Azapodophyllotoxin composition having the general formula: 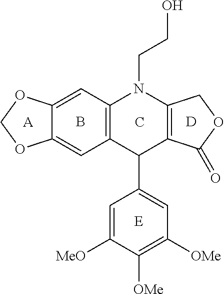
wherein a compound is selected from the group consisting of APA-107, APA-108, APA-109, APA-207, APA-208, APA-209, APA-307, APA-308, APA-309, APA-401, APA-402, APA-403, APA404, APA-405, APA-406, APA-407, APA-408, APA-409, APA-501, APA-502, APA-503, APA-504, APA-505, APA-506, APA-507, APA508, APA-509, APA-601, APA-602, APA-603, APA-604, APA-605, APA-606, APA-607, APA-608, and APA-609, as shown in FIG. 4 of the drawings; comprising the steps of:
a. combining tetronic acid, substituted aniline, and an aromatic aldehyde in equimolar proportions, dissolved in a minimal volume of ethanol;
b. irradiating the resulting mixture from the previous step with microwaves using approximately 6 watts for approximately forty five minutes;
c. allowing for the cooling of the mixture;
d. filtering the precipitate off;
e. washing the precipitate with a minimal volume of cold ethanol; and
f. recrystallizing the precipitate from ethanol.

29. A synthesis procedure for a Azapodophyllotoxin composition having the general formula: 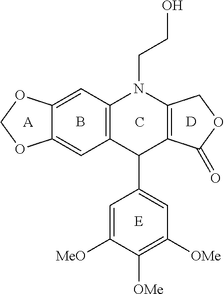
wherein a compound is selected from the group consisting of APA-107, APA-108, APA-109, APA-207, APA-208, APA-209, APA-307, APA-308, APA-309, APA-401, APA-402, APA-403, APA404, APA-405, APA-406, APA-407, APA-408, APA-409, APA-501, APA-502, APA-503, APA-504, APA-505, APA-506, APA-507, APA508, APA-509, APA-601, APA-602, APA-603, APA-604, APA-605, APA-606, APA-607, APA-608, and APA-609, as shown in FIG. 4 of the drawings; comprising the steps of:
combining tetronic acid, substituted aniline, and an aromatic aldehyde in equimolar proportions, dissolved in a minimal volume of ethanol;
irradiating the resulting mixture from the previous step with microwaves using approximately 6 watts for approximately forty five minutes;
allowing for the cooling of the mixture;
filtering the precipitate off;
using silica column chromatography to purify the precipitate; wherein said column chromatography comprises a slurry of dry silica gel 60 and 230-400 mesh solid support;
and wherein a mixture of hexane:ethyl acetate:acetonitrile in 40:40:20 proportion is used as eluent.
